(19) United States
(12) Patent Application Publication
(10) Pub. No.: US 2023/0108528 A1
(43) Pub. Date: Apr. 6, 2023

(54) ARTIFICIAL SYNTHETIC MACROCYCLE MOLECULAR NANOPORE STRUCTURES AND PREPARATION METHODS AND APPLICATIONS

(71) Applicant: Zhejiang University, Hangzhou (CN)

(72) Inventors: Jiandong Feng, Hangzhou (CN); Dan Qiao, Hangzhou (CN)

(21) Appl. No.: 17/963,165

(22) Filed: Oct. 10, 2022

Related U.S. Application Data
(63) Continuation of application No. PCT/CN2021/099256, filed on Jun. 9, 2021.

(51) Int. Cl. G01N 33/487; G01N 27/04; C12Q 1/6869

(52) U.S. Cl. CPC G01N 33/48721; G01N 27/041; C12Q 1/6869

(57) ABSTRACT

The present invention belongs to the field of bioanalysis and detection, specifically, a synthetic macrocyclic molecular nanopore structure and preparation method and application. The invention discloses an artificially synthesized macrocyclic compound to form a stable single-molecule nanopore structure on phospholipid bilayer; the nanopore structure is a transmembrane nanopore structure with nano-sized channels formed by the artificially synthesized macrocyclic compound inserted into the phospholipid bilayer membrane in electrolyte solution; the artificially synthesized macrocyclic compound solves the transmembrane nanopore cavity size and pore thickness by using the bottom up synthesis, which yields thinner pore thickness and higher freedom control of the cavity pore size compared with the traditional biological nanopores constructed by proteins.

### CROSS-REFERENCE TO RELATED APPLICATIONS

The present application is a continuation application of PCT application No. PCT/CN2021/099256 filed on Jun. 9, 2021, which claims the benefit of Chinese Patent Application No. 202010278865.X filed on Apr. 10, 2020, each of which is incorporated by reference herein in its entirety.

### TECHNICAL FIELD

The present invention belongs to the field of bioanalysis and detection, specifically, an artificial synthetic macrocycle molecular nanopore structure and preparation method and application.

### BACKGROUND

Transmembrane nanopores have become a powerful tool for chemical and biological sensing, and have also achieved remarkable success in DNA sequencing. Transmembrane nanopores can be self-assembled from a variety of structures, including proteins, peptides, synthetic organic compounds, and DNA origami. Compared to solid-state nanopores, transmembrane nanopores can be better compatible with applications involving vesicles and cells as well as membrane-based analytical platforms by inserting lipid bilayers. In addition, transmembrane nanopores are reproducible for detection and DNA sequencing due to the homogeneous protein structure. However, most of the protein nanopores used in current nanopore technologies suffer from low resolution and sensitivity. This is due to the fact that the effective thickness or sensing length of conventional biological nanopores is greater than 2 nm, which leads to the identification of four of the best currently disclosed protein nanopores for DNA sequencing (e.g. MspA pores, Laszlo, A., Derrington, I., Ross, B. et al. Decoding long nanopore sequencing reads of natural DNA. Nat Biotechnol 32, 829-833 (2014). https://doi.org/10.1038/nbt.2950) or more nucleotide combination sequences that do not provide direct single-base resolution, greatly compromising the spatial resolution of nanopore technology and limiting further applications of the current nanopore technology as it evolves from DNA sequencing to protein sequencing.

Macrocyclic compounds first appeared in 1890 and have also recently sparked a boom in supramolecular chemistry research and development. They include pillar[n]arenes, crown ethers, calix[n]arenes, Cucurbit[n]urils, cyclodextrins, etc. Most of the types of macrocyclic compounds to date are listed in the following collection of macrocyclic compound structures, where the pore size can reach 10 nm or more, such as pillar[n]arenes or derivatives of aromatic acetylene planar rigid macrocycles composed by ring-opening synthesis. Supramolecular chemistry has a wide range of applications in various fields, such as molecular recognition, sensing, molecular machines and devices, supramolecular polymers, excited-state responsive materials, supramolecular catalysis, and drug delivery systems. The unique structure of macrocyclic compounds makes it possible to apply them to transmembrane nanopore as well.

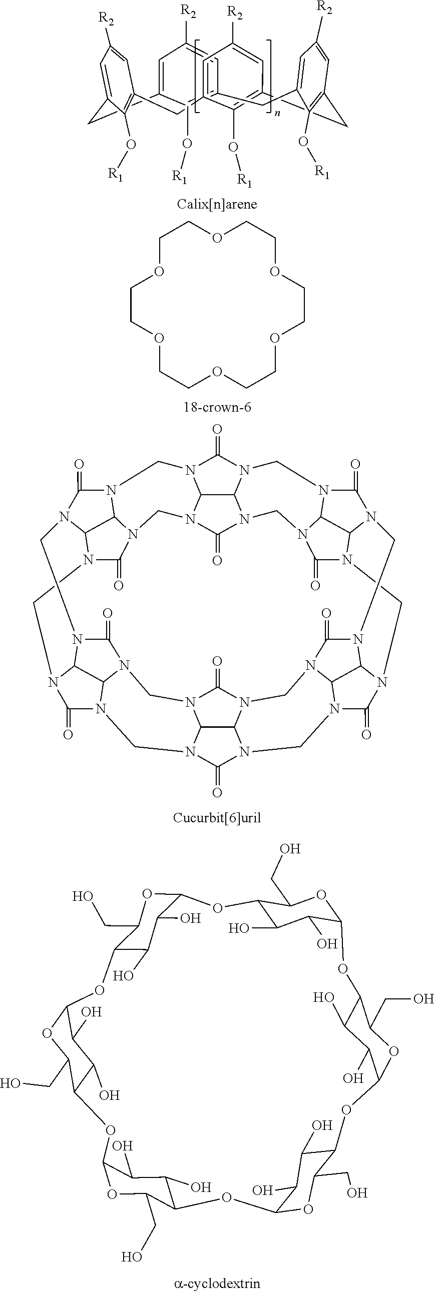

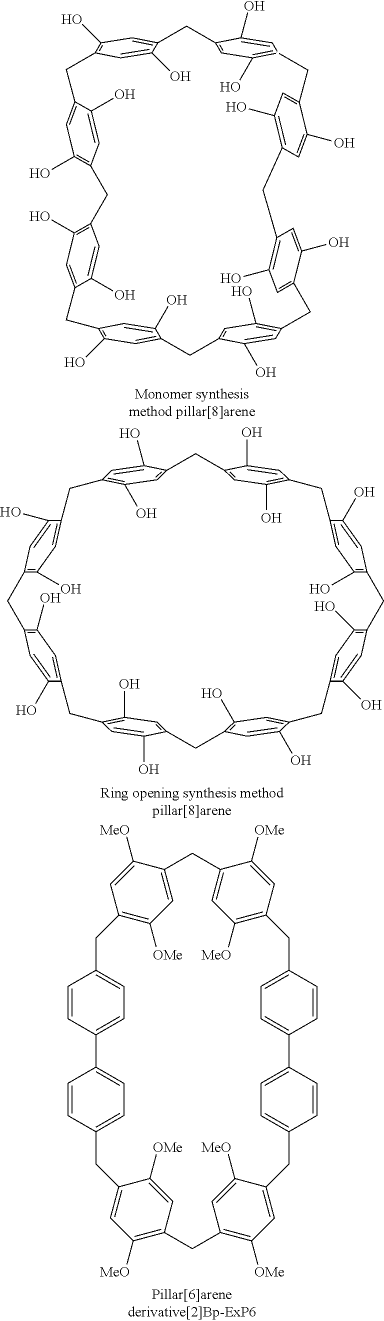 [SMILES: CCc1cc(O)c(Cc2cc(O)c(Cc3cc(O)c(Cc4cc(O)c(Cc5cc(O)c(Cc6cc(O)c(Cc7cc(O)c(Cc8cc(O)ccc8O)cc7O)cc6O)cc5O)cc4O)cc3O)cc2O)cc1O ; Oc1cc2c(O)cc1Cc1cc(O)c(cc1O)Cc1cc(O)c(cc1O)Cc1cc(O)c(cc1O)Cc1cc(O)c(cc1O)Cc1cc(O)c(cc1O)Cc1cc(O)c(cc1O)Cc1cc(O)c(cc1O)C2 ; COc1cc2c(OC)cc1Cc1ccc(cc1)-c1ccc(cc1)Cc1cc(OC)c(cc1OC)Cc1cc(OC)c(cc1OC)Cc1ccc(cc1)-c1ccc(cc1)Cc1cc(OC)c(cc1OC)C2]

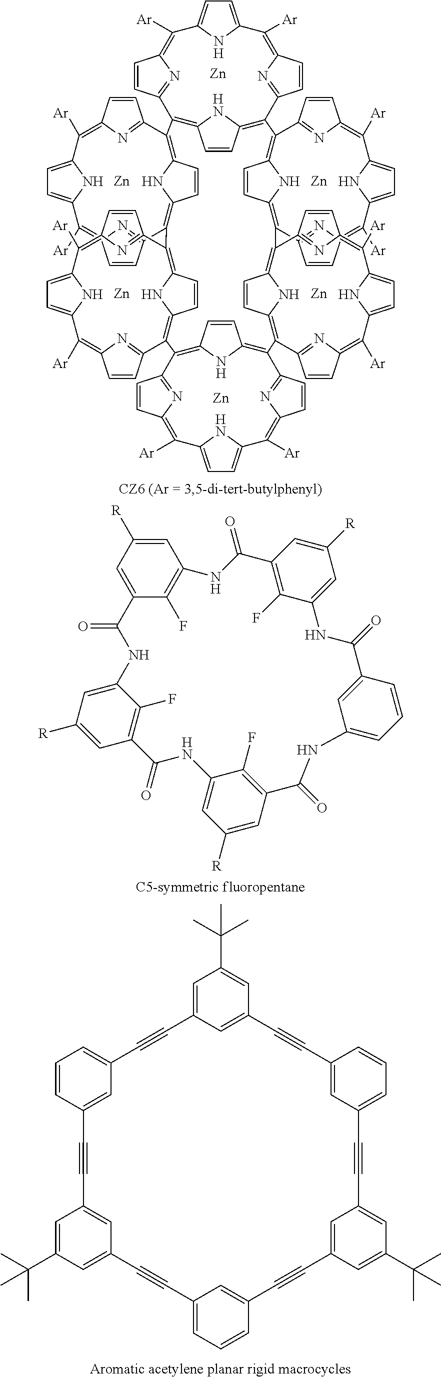

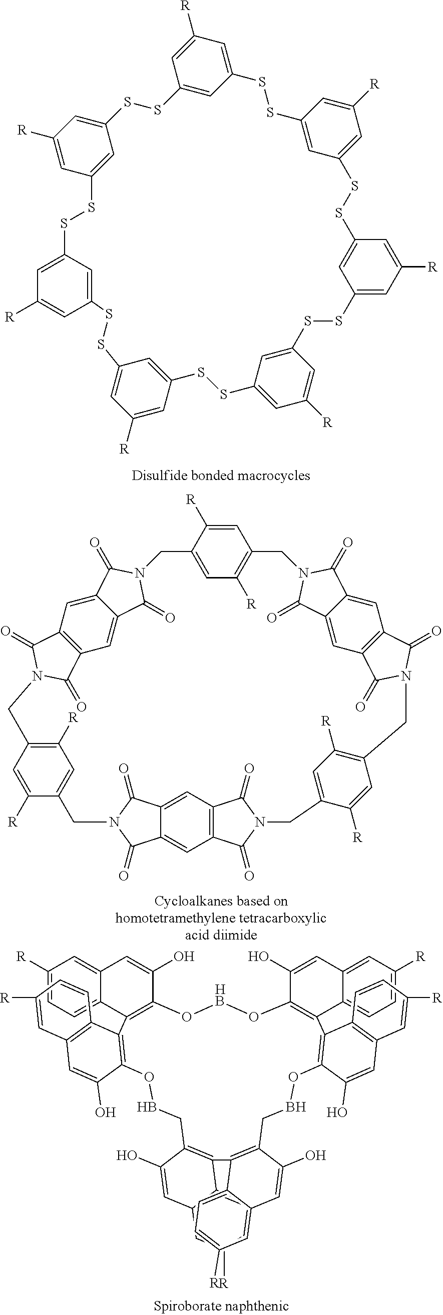

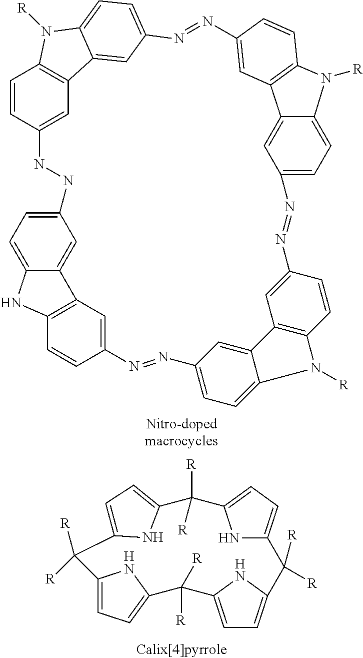

The core design of the present invention is the use of organically synthesized macrocyclic structures as transmembrane nanopores. The macrocyclic molecular transmembrane nanopore can be designed with a greater degree of modulation of pore size, dynamics, and interactions with other molecules at the atomic precision level by simple design. In addition to this, the pore thickness and size in these synthetic transmembrane nanostructures can be tuned by design down to the size of a single nucleotide or amino acid, and thus they can provide the necessary atomic-level spatial resolution for nanopore DNA sequencing or even protein sequencing. In addition, transmembrane nanopores offer great advantages and greater possibilities in terms of chemical, structural and nanomechanical tunability.

To date, most nanopore sensing studies have used pore-forming transmembrane proteins containing β-barrel types with hydrophobic surfaces. Because these proteins are more easily inserted into planar lipid bilayer membranes, this makes them perfect candidates for sensing applications such as aerosolysin, α-hemolysin. However, other non-β-barrel type proteins or synthetic transmembrane nanostructures may also provide superior analyte recognition properties, but sensing experiments using this type of nanopore are influenced by the ability to stably insert into lipid membranes.

For example, hydrophilic pore-containing structures, such as the synthetic macrocyclic structures designed by the present invention, require appropriate chemical modifications to give them lipid anchors (hydrophobic bands) to make them easier to insert into phospholipid bilayer membranes to form transmembrane nanopores. Chemical modifications for such nanopore structures are commonly used, for example, porphyrins, cholesterol, ethyl phosphorothioate (EP), tocopherols, long alkane chains, or anchors formed by linking multiple polypeptides, etc. The specific modified structures are shown in Table 1 below.

TABLE 1
Types of side chain modifications and structural formulae of transmembrane
nanopores

| Types of side | |
|---|---|
| chain | Structure |
| Porphyrins | 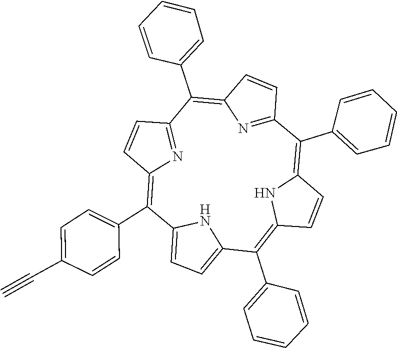 [SMILES: C#Cc1ccc(-c2c3nc(c(-c4ccccc4)c4nc(c(-c5ccccc5)c5ccc([nH]5)c(-c5ccccc5)c5ccc2[nH]5)C=C4)C=C3)cc1] |
| | 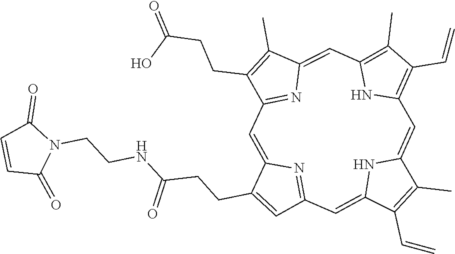 [SMILES: C=Cc1c(C)c2cc3[nH]c(cc4nc(cc5nc(cc1[nH]2)C=C5CCC(=O)NCCN1C(=O)C=CC1=O)C(CCC(=O)O)=C4C)c(C)c3C=C] |
| | 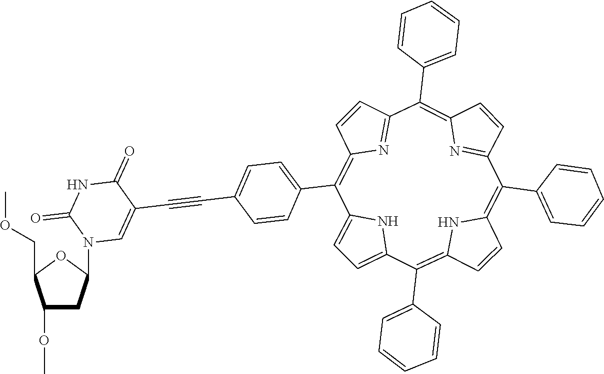 |
| Cholesterol | 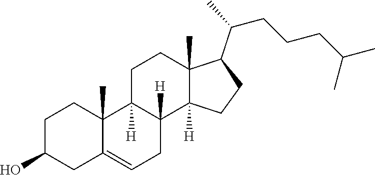 |
| | 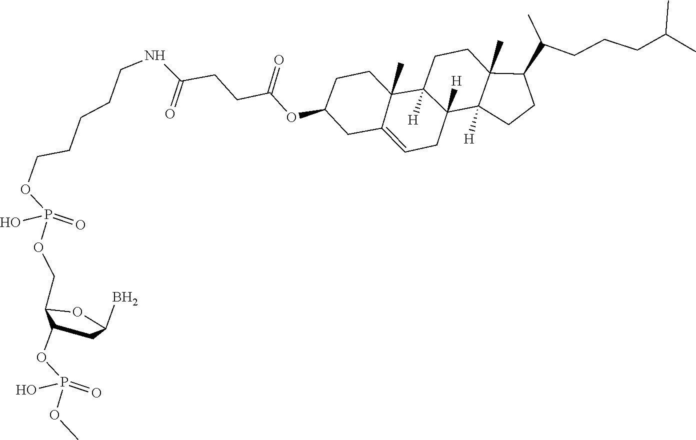 |
| EP | 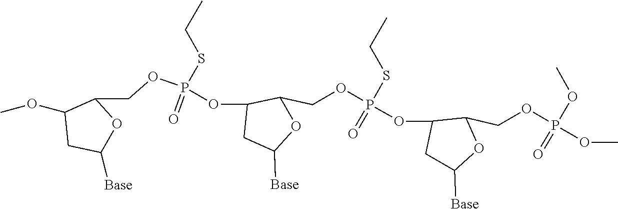 |
| Tocopherols | 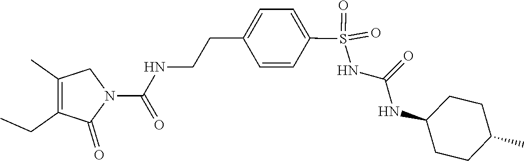 [SMILES: CCC1=C(C)CN(C(=O)NCCc2ccc(S(=O)(=O)NC(=O)N[C@H]3CC[C@H](C)CC3)cc2)C1=O] |
| Alkane chains | 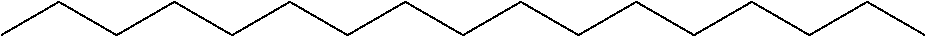 [SMILES: CCCCCCCCCCCCCCCCC] |
| Peptide chains | 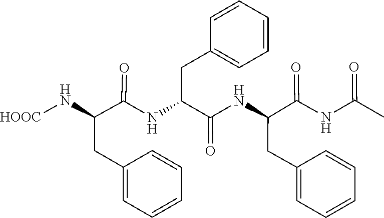 [SMILES: CC(=O)NC(=O)[C@@H](Cc1ccccc1)NC(=O)[C@@H](Cc1ccccc1)NC(=O)[C@@H](Cc1ccccc1)NC(=O)O] |

Other non-β-barrel transmembrane nanostructures, such as pore-containing polar proteins, can also be modified with porphyrins to make them stable in lipids.

Therefore, structurally, these macrocyclic structures have some similarity to conventional biological nanopore structures, but can achieve higher precision structural control and theoretical spatial resolution of single nucleotides or single amino acids, and the required chemical synthesis and modification schemes also have significant batch preparation and cost advantages compared to conventional protein nanopore preparation. The application of macrocyclic compounds to transmembrane nanopore structures for ion transport applications or biomolecule detection or sequencing has great promise and application value.

### SUMMARY

The present invention discloses an artificial synthetic macrocycle molecular nanopore structures and preparation methods and applications, wherein:

The present invention discloses a synthetic macrocyclic compound to form a stable single-molecule nanopore structure on phospholipid bilayer membrane; the nanopore structure is a transmembrane structure with nano-sized channels formed by the insertion of the synthetic macrocyclic compound into the phospholipid bilayer membrane in electrolyte solution; the artificially synthesized macrocyclic compound solves the transmembrane nanopore cavity size and pore thickness by using the bottom up synthesis, which yields thinner pore thickness and higher freedom control of the cavity pore size compared with the traditional biological nanopores constructed by proteins. Macrocyclic compounds generally have cavities with diameters of 1 Å-50 Å. As mentioned above, the pore size of pillar[n]arene compounds or aromatic acetylene planar rigid macrocyclic derivatives composed by ring-opening synthesis can even reach 10 nm or more. Nanopores formed by macrocyclic compounds with diameters of 1 Å-15 Åcan be used for selective ion transport, with diameters greater than 12 Å for DNA single-strand sequencing, pore diameters greater than 24 Å for DNA double-strand sequencing, and diameters greater than 8 Å for protein sequencing and protein recognition detection; the cavities of macrocyclic compounds have atomic-level thicknesses of 1 Å-30 Å, and after forming nanopores, the DNA or biomolecules such as proteins pass through the cavity, improving the spatial resolution. For other needs of larger sizes, further larger cavity structures can also be used.

As a further improvement, the synthetic macrocyclic compound has side chains that help insert the macrocyclic molecule into the phospholipid membrane to facilitate the formation of a stable transmembrane structure.

As a further improvement, the side chains are linked to the macrocycles by amide or ether bonds or carbon-carbon bonds.

As a further improvement, the synthetic macrocyclic compound is a cucurbiturate derivative or a cyclodextrin derivative or a crown ether derivative or a macrocyclic compound derivative consisting of an aromatic hydrocarbon.

As a further improvement, the synthetic macrocyclic compound is the pillar[6]arene derivative called EPM, the molecular formula of EPM is C₃₇₄H₃₈₈N₄₀O₅₆ and the structural formula of EPM is:

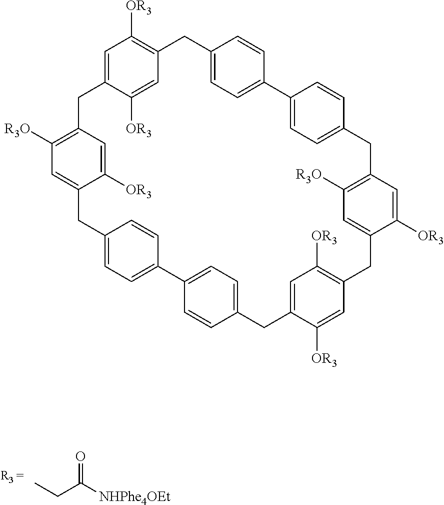

The present invention also discloses a method for the preparation of synthetic macrocyclic compounds to form stable single-molecule nanopore structures on phospholipid bilayer membranes, comprising the following steps:
1) synthesizing the pillar[6]arene derivative by a chemical method;
2) preparing a perfusion cup for constructing the phospholipid bilayer membrane and performing ion channel experiments, wherein:
   the perfusion cup is separated into a cis-side chamber and a trans-side chamber by a cup wall, wherein the cup wall has a support hole, and the phospholipid bilayer membrane is built on the support pore and then inserted into the synthetic macrocyclic compound to form the nanopore structure;
3) dissolving the synthetic macrocyclic compound in water or a buffer solution, sonicating, filtering undissolved material, dividing the filtered solution into aliquots, freezing, and storing the frozen aliquots;
4) polishing two pieces of silver wire with a sandpaper to remove an oxide layer on the surface of the silver wire, immersing the silver wire and a platinum electrode in a plating solution, the silver wire and the platinum electrode serving as the anode and cathode respectively, applying a voltage to prepare a silver/silver chloride electrode, and then connecting two silver/silver chloride electrodes to probes of a patch clamp instrument as anode and ground wire respectively;
5) preparing a lipid solution;
6) applying the lipid solution uniformly to both sides of the support hole of the perfusion cup using a brush until the support hole is uniformly covered and waiting for the lipids to dry at room temperature;
7) pipetting the electrolyte solution into each of the cis-side chamber and the trans-side chamber at a time;
8) performing the following steps in a Faraday box on an optical platform: immersing the silver/silver chloride electrodes serving as anode and ground wire in the electrolyte solution of the cis-side chamber and the trans-side chamber, respectively; and
   turning on the patch clamp instrument, applying a positive potential to the trans side through the silver/silver chloride electrode, and grounding the cis side;
9) using a pipettor to lift a solution interface up and down on both sides of the support hole, such that a lipid monolayer formed by the lipid solutions on both sides forms a phospholipid bilayer membrane due to the hydrophobicity of hydrocarbon chains of the phospholipid molecules;
10) determining the phospholipid bilayer membrane as a bilayer structure by measuring the capacitance of the phospholipid bilayer membrane or by applying a membrane breaking voltage; and
11) thawing the frozen aliquots in step 3) by ultrasonication and then diluting the thawed aliquots using deionized water with 1 wt % of non-ionic surfactant; and
   adding a solution of the synthetic macrocyclic compound very close to the support hole in the cis-side chamber, applying a voltage, and when a step jump in current occurs, it indicates that the synthetic macrocyclic compound has formed stable nanopore channels in the phospholipid bilayer membrane.

As a further improvement, an outer chamber of the perfusion cup is provided with small holes connected to its inside chamber; the lipid solution is one selected from the group consisting of 1,2-diacetyl-sn-glycero-3-phosphocholine, palmitoyl oleoyl phosphatidylcholine, dipalmitoyl phosphatidylcholine, and distearoyl phosphatidylglycerol dissolved in decane;

in step 10), a capacitance value is 35-90 pF when the phospholipid bilayer membrane is a bilayer structure; or when the phospholipid bilayer membrane is a bilayer structure, the phospholipid bilayer membrane is broken within an applied potential of 300-400 mV; and

in step 11), avoiding any air bubbles when adding a solution of synthetic macrocyclic compound; and when a step jump in current occurs, the voltage is reduced in time.

As a further improvement, the perfusion cup and the cup wall are made of polyformaldehyde resin, polytetrafluoroethylene, or polystyrene. The materials mentioned are all hydrophobic materials, due to the hydrophobic nature of the hydrocarbon chains at the end of the phospholipid molecules, two lipid monolayers form a phospholipid bilayer, so the perfusion cup material is hydrophobic making it easier for the phospholipid bilayer to form on both sides of the support hole.

The present invention also discloses the application of a synthetic macrocyclic compound forming a stable single-molecule nanopore structure on a phospholipid bilayer in the artificial construction of ion channels, applying the structure to achieve efficient selective transport and separation of potassium ion/sodium ion.

The invention also discloses the application of a synthetic macrocyclic structured molecular nanopore structure for biomolecule detection or sequencing, applying the structure to achieve protein peptide sequencing, or detection and sequencing of similar biomolecule or chemical molecule based on the same principle.

The present invention also discloses the application of a synthetic macrocyclic structured molecular nanopore structure for biomolecule detection or sequencing, applying the structure to achieve DNA sequencing or RNA sequencing.

The synthesized macrocyclic compounds of the present invention offer great advantages and greater possibilities in terms of chemical, structural and nanomechanical tunability, and thus their application to nanopore aspects provides the necessary spatial resolution for nanopore DNA sequencing and even protein sequencing.

The beneficial effects of the present invention:
1) The designed synthetic macrocyclic compound forms a stable single molecule structure uniquely on the phospholipid bilayer, which is different from all common transmembrane nanopores, the cavity thickness is only at the atomic level, the chemical structure is uniform, the chemical properties are stable, and a stable single nanopore channel can be formed with high experimental reproducibility and uniformity.
2) Compared with other artificial nanopores with potassium ion selectivity, EPM has relatively high potassium ion selectivity without additional chemical modifications, with a potassium ion/sodium ion selectivity factor as high as 20.
3) There are many types of macrocyclic molecules with cavity structures, and the present invention takes EPM molecules as a typical example to demonstrate that these macrocyclic molecules can form stable nanopore channels, and the cavity thickness of synthetic macrocyclic compounds is atomic level compared with other transmembrane nanopores, which provides higher spatial resolution for protein sequencing.
4) The synthetic macrocyclic molecule is a synthetic chemical structure with abundant modification sites on the cavity, so it can have a wide selection of modification groups to make it have more different chemical properties, which provides a higher possibility for its application as a nanopore.
5) The cavity size of the synthetic macrocyclic molecules can be freely adjusted according to the number of benzene rings, which provides the possibility of applying them to DNA sequencing after the expansion of the pores.
6) The experimental operation is simple and can be applied without unnecessary modifications or other post-processing.

### BRIEF DESCRIPTION OF THE DRAWINGS

FIG. 1 shows the specific structure, size and MALDI-TOF mass spectrometry results of the EPM molecule, wherein:

a. 3D geometry of the synthetic EPM nanopores. b. Chemical structure diagram of EPM nanopore. c. MALDI-TOF mass spectrum of the EPM pore. d. Cryo-EM of EPM nanopores on lipid vehicles. Scale bar: 20 nm.

FIG. 2 shows a schematic diagram of the perfusion cup and the entire nanopore device, wherein:

1 is silver/silver chloride electrode, 2 is the support hole. 3 is cis side and 4 is trans side. 5 is Electrolyte solution. 6 is the phospholipid bilayers. 7 is pillar[6]arene derivatives of macrocyclic compounds, EPM. 8 is the cup wall.

FIG. 3 shows results of single channel recordings of ion transport through individual EPM nanopores, wherein:

a. Stepwise incorporation of individual EPM nanopores into lipid bilayers at −120 mV in a 500 mM KCl solution. b. Schematic diagram of EPM nanopore structure. c. Histogram of channel conductance obtained from 94 single-step incorporation events. d. Current—voltage characteristic of a single EPM nanopore. e. Typical current traces and normalized current histograms (right) of individual EPM nanopores, and the gating behavior. The normalized histogram of the current trajectories generated by the stable nanopores is unimodal. The normalized histogram of the current trajectory with gating behavior is bimodal, indicating that the nanopore switches between two conductance states.

FIG. 4 shows results of potassium ion selectivity of EPM nanopores, wherein:

a-c. I-V plots of individual EPM nanopores in different concentrations of potassium chloride/sodium chloride solutions. d. Potassium ion selectivity of EPM nanopores versus ionic strength. e. I-V plots of individual EPM nanopores in mixed solutions. f. Potassium ion selectivity of individual EPM nanopores in mixed solutions versus potassium ion concentration in mixed solutions percentage plot.

FIG. 5 shows comparative results of potassium ion selectivity of EPM nanopores, wherein:

a-c. I-V plots in different concentration gradients of potassium chloride and sodium chloride solutions. d. I-V plots in 1 M potassium chloride-1 M sodium chloride solution.

FIG. 6 shows results of current blocking events and current traces recordings, wherein:

a. Plot of dwell time versus current blockage value for short peptide chain GG passing through EPM nanopores at 100 mV. b. Plot of dwell time versus number of current blockage events for short peptide chain GG passing through EPM nanopores at 100 mV. c. Plot of dwell time versus current blockage value for short peptide chain GG passing through EPM nanopores at 120 mV. d. Plot of dwell time versus number of current blockage events for short peptide chain GG passing through EPM nanopores at 120 mV

### DETAILED DESCRIPTION

### Examples

In this example, a pillar[6]arene derivative macrocyclic molecule EPM with amphiphilic side chains was synthesized artificially. Using the characteristics of EPM, a single molecular channel experiment of transmembrane nanopore on phospholipid bilayer (6) was successfully completed based on Axonpatch instruments, and a series of experiments were conducted with the premise of single molecular channel, and it was found that the EPM nanopore has a potassium ion selectivity with a selection factor as high as 20. In addition, it was demonstrated experimentally that the nanopore formed by EPM has the potential for protein sequencing and the possibility of application to DNA sequencing.

Pillar[6]arene derivative macrocyclic molecule EPM with amphiphilic side chains has only one large rigid cavity, and better stability and larger cavity, the theoretical diameter, i.e., the maximum distance between atoms, is about 12 Å, which is twice the diameter of the cavity of common pillar[5]arene and pillar[6]arene. The side chains selected in this example are four phenylalanines connected to one ester ethyl, modified on a total of eight sites at the upper and lower ends of the EPM molecule, making it has certain lipophilic hydrophilic at the same time, and the length of the side chains with four phenylalanines in each of the upper and lower layers is about the same as the thickness of phospholipid bilayer (6) (5 nm), which increases the stability of molecules forming transmembrane nanopores.

A synthetic macrocyclic compound pillar[6]arene derivative EPM forms a stable single-molecule nanopore structure on phospholipid bilayer (6), a transmembrane structure with nano-sized channels formed by the insertion of the synthetic macrocyclic compound into the phospholipid bilayer (6) membrane in an electrolyte solution; the macrocyclic compound has an atomic-level thickness of 1 Å-30 Å and side chains that help insert the macrocyclic molecules into the phospholipid membrane. EPM has the molecular formula C₃₇₄H₃₈₈N₄₀O₅₆ and the structural formula is:

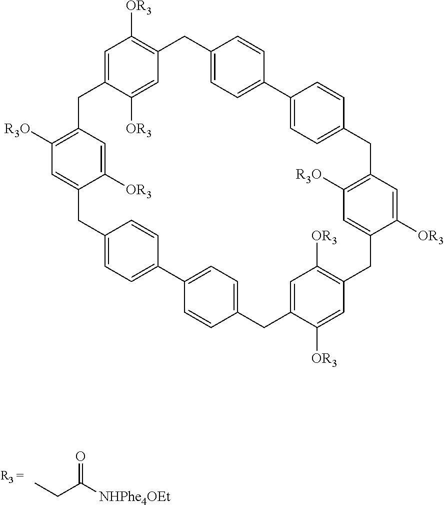

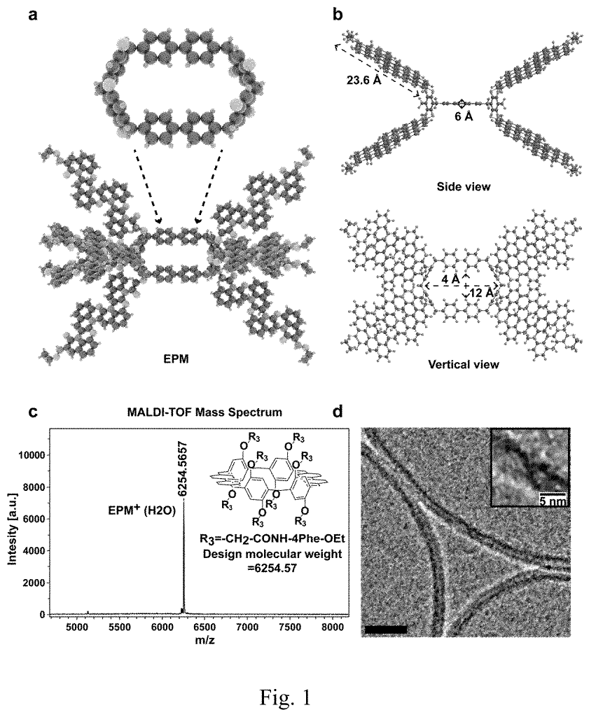

The specific structure, size and MALDI-TOF mass spectrometry results of the EPM molecule are shown in FIG. 1. a in FIG. 1 shows the schematic 3D geometry of the EPM molecule, b in FIG. 1 shows the chemical structure of the EPM nanopore, c in FIG. 1 shows the MALDI-TOF mass spectrometry results of the EPM molecule, and d in FIG. 1 shows the cryo-electron microscopy results of the EPM molecule combined with a bilayer phospholipid vesicle, scale bar is 20 nm.

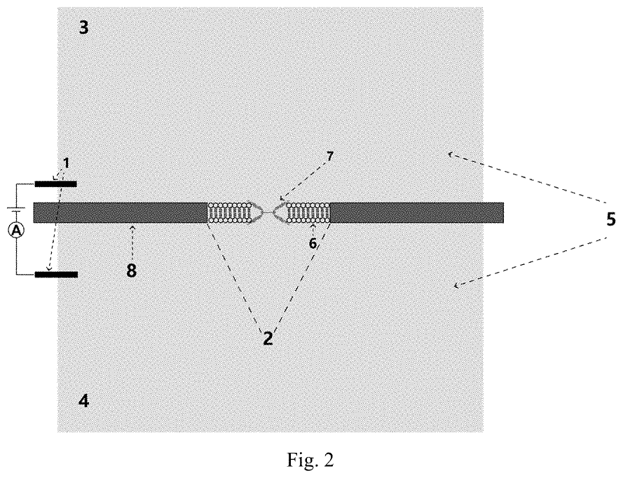

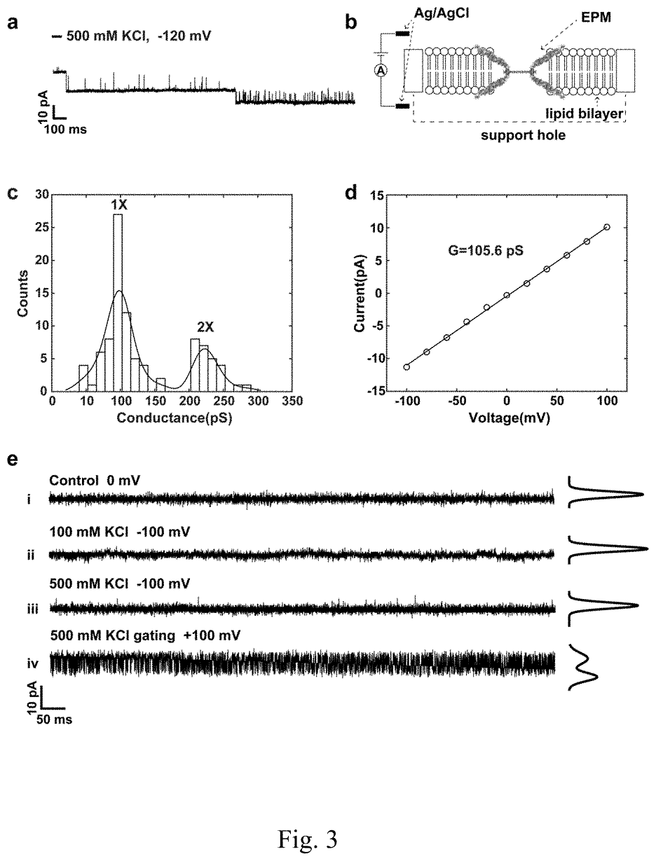

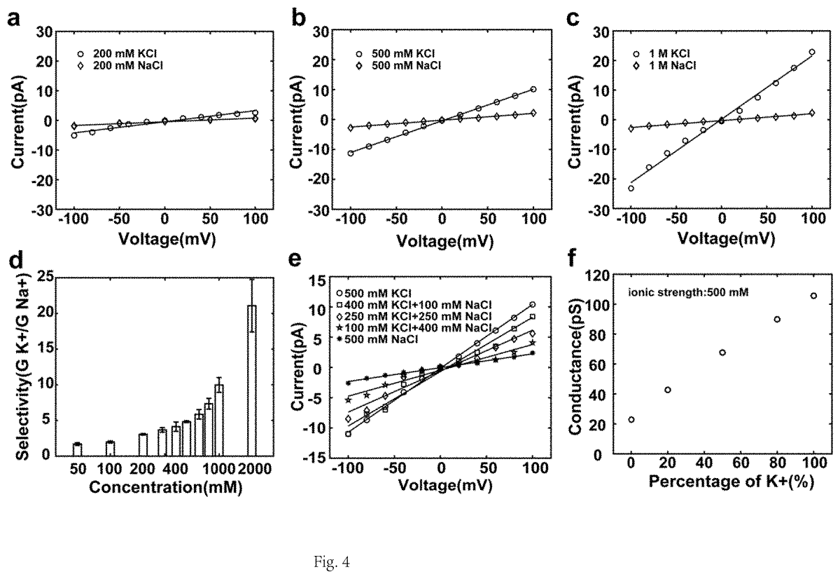

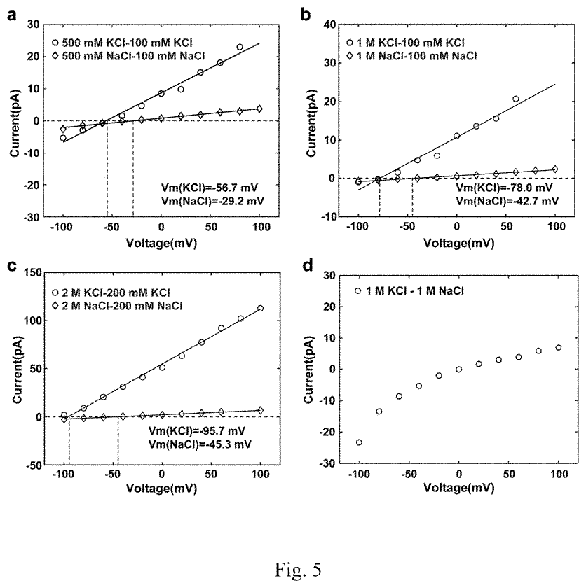

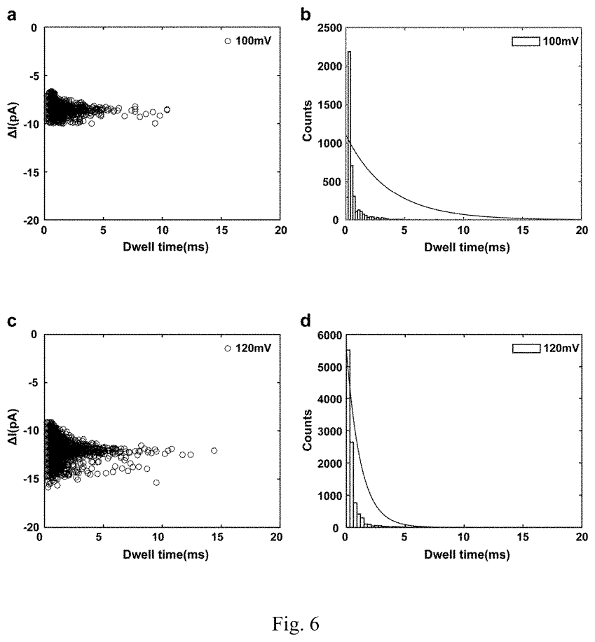

Pillar[6]arene derivative macrocyclic compound EPM (7) was prepared as follows:
1) Synthesis of pillar[6]arene derivatives of macrocyclic molecules EPM by chemical methods, wherein:
   Aluminum chloride was added to a solution of 4,4′-bis(chloromethyl)-1,1′-biphenyl and 1,4-diethoxybenzene in dichloromethane. After the reaction, the organic phase was separated and concentrated, purified by column chromatography and recrystallized to obtain the pure product in the form of a white solid. The above product and oligomeric formaldehyde were stirred in trichloromethane, then BF₃-OEt₂ was injected and purified on column chromatography after reaction to give the pure product as a white solid. The white solid pure product was dissolved in trichloromethane under the protection of argon, and then an excess of BBr₃ was added. The precipitate was filtered after the reaction. The above precipitate, potassium carbonate and ethyl 2-bromoacetate were dissolved in acetonitrile under argon protection, and the suspension was filtered and the filtrate concentrated. The product was purified on column chromatography to give a white solid pure product of pillar[6]arene derivatives with substituent —CH₃COOCH₂CH₃.
   The above product, the pillar[6]arene derivative whose substituent is —CH₃COOCH₂CH₃, was suspended in a mixture of water and ethanol. After the addition of sodium hydroxide, the mixture was refluxed overnight. The homogeneous solution was poured into hydrochloric acid; then it was poured into water and filtered to obtain a white precipitation product. The above white precipitate, H2N-Phe-Phe-Phe-Phe-OEt, 4-dimethylaminopyridine and 1-ethyl-(3-dimethylaminopropyl) carbodiimide hydrochloride were suspended in dimethylformamide and the suspension was poured into hydrochloric acid. The product was filtered and dried. Due to multiple amide bonds, the product could not be purified by conventional methods and the mixture was used directly in the following experiments. MALDI-TOF mass results indicate that pillar[6]arene derivative macrocyclic molecule EPM (7) with substituent —CH₂—CO—NH-Phe-Phe-Phe-Phe-OEt has been successfully prepared: measured value 6254.57. MALDI-TOF spectra of other peaks can be attributed to partial peptide substitution of macrocycles and amide hydrolysis. The specific structure, dimensions and MALDI-TOF mass spectrometry results are shown in FIG. 1.
2) Design of perfusion cup, wherein:
   FIG. 2 shows a schematic diagram of the perfusion cup and the whole nanopore device; the perfusion cup is used for ion channel experiments on phospholipid bilayer (6), and a perfusion cup with a support hole (2) is used as a base to build the phospholipid bilayer (6) membrane on the support hole (2), and then synthetic macrocyclic molecules are used to form nanopores. The perfusion cup is divided into two chambers, cis side (4) and trans side (3), by the cup wall (8), and the two chambers are used to carry electrolyte solution (5), and silver/silver chloride electrode (1) is placed in each of the two chambers.
3) Dissolution of EPM (7) molecules, wherein:
   Take 1 mg of EPM (7) molecule dissolved in 10 mL of water, ultrasonically dissolve and filter the undissolved substance, then store the molecular solution in −80° C. refrigerator in portions, and take a certain amount for ultrasonic thawing before each experiment.
4) Preparation of electrodes and connection of instruments, wherein:
   Two lengths of silver wire of about 3 cm were taken and polished with sandpaper to remove the oxide layer on the surface. Immerse three-fourths of the length of the silver wire and the platinum electrode (as anode and cathode, respectively) in a 1 M potassium chloride solution. A voltage of 3 V or less is applied for a period of time to prepare the silver/silver chloride electrode (1). The two silver/silver chloride electrodes (1) are then connected to the probe of the current amplifier Axopatch 200B of the digital-to-analog converter DigiData 1550B as the positive and ground wires, respectively.
5) Preparation of lipid solutions, wherein:
   A lipid solution at a concentration of 30 mg/mL was prepared by dissolving 3 mg of 1,2-diacetyl-sn-glycero-3-phosphocholine in 100 uL of decane. The solution can be stored at 4° C. for one week and should be reconstituted after one week.
6) Application of lipid solutions, wherein:
   Pipette 1 uL of lipid solution onto the tip of a 000-bristle pen using a pipettor, and then apply the solution evenly to both sides of support hole (2) until the hole is evenly covered. Wait a few minutes at room temperature to dry the lipids.
7) Addition of electrolyte solutions (5), wherein:
   1 mL of potassium chloride or 1 mL of sodium chloride solution is pipetted into each of the two chambers, cis side (4) and trans side (3).
8) Placement of experimental setup and instrument opening, wherein:
   The entire device is placed in a Faraday box on an optical stage to avoid vibration and electrical interference and to keep the single-channel current recording low noise. Immerse the positive and ground silver/silver chloride electrodes (1) in the solution of the cis (4) and trans (3) chambers, respectively.
   A positive potential is applied to the trans side (3) (cis side (3) ground) using Axonpatch instrument and through a silver/silver chloride electrode (1).
9) Construction of phospholipid bilayer (6), wherein:
   The electrolyte solution (5) on the cis side (4) is slowly moved below the aperture of the support pore (2) using a 1000 uL size pipettor, and the ionic current drops to 0 when the air-electrolyte solution (5) interface is below the support hole (2), and then the electrolyte solution (5) is slowly moved horizontally above the support pore (2). Due to the hydrophobicity of the hydrocarbon chains of phospholipid molecules, two lipid monolayers will begin to form a phospholipid bilayer (6) film.
10) Identification of phospholipid bilayer (6) membrane as a bilayer structure, wherein:
   For a 150 um support hole (2), the capacitance value of the phospholipid bilayer (6) membrane should be in the range of 35-90 pF, depending on the experimental setup and environment, and should be figured out during the experiment. The capacitance of the phospholipid bilayer (6) membrane was measured using a Axonpatch instrument, and if the capacitance is greater than this range, it indicates that the lipid membrane is too thin, at which point the lipid solution (<1 uL) should be made to be taken at the tip of the brush and applied to the support hole (2). If the capacitance is less than that range, the membrane is too thick and another clean brush should be taken to brush the support hole (2) until the membrane breaks (the current is no longer 0), and then the phospholipid bilayer (6) membrane should be re-formed as in step 9) until the capacitance value is in the appropriate range.
   Phospholipid membranes can be tested for bilayers by applying a voltage. If the formed phospholipid membrane can be broken within an applied potential of 300-400 mV, the membrane is re-formed under step 9) above, and after re-formation, the phospholipid bilayer (6) membrane has the appropriate pore insertion thickness.
11) Insertion of a single synthetic macrocyclic compound molecule into a phospholipid bilayer (6) membrane, wherein:
   The stock solution of EPM molecules were sonically thawed and then diluted using ultrapure water with 1% wt of a nonionic surfactant (polyethylene glycol monoolether) to make the molecules more dispersed and prevent aggregation.
   A solution of 40-50 uL EPM molecules is added in the cis side (3) chamber very close to the support pore (2), a voltage is applied and when a jump in current occurs it indicates that the EPM (7) molecules have formed stable nanopore channels in the phospholipid bilayer (6) membrane. Be careful not to add any air bubbles when adding the molecular solution to prevent the lipid bilayer from becoming unstable and breaking.
   Note the observation that when a stepwise jump in current occurs, the voltage should be reduced in time because the high potential may lead to the possibility of a second molecular insertion while we aim to observe on the basis of a single molecular channel. The current trajectory of the final EPM (7) molecules inserted one by one into the phospholipid membrane to successfully form nanopores is shown in a in FIG. 3, where the uniform stepwise current jumps indicate the sequential insertion of individual EPM (7) molecules into the phospholipid bilayer (6) membrane to form nanopores. Based on extensive experience, each current step corresponds to a single nanopore embedded in the phospholipid bilayer (6), forming the nanopore structure schematically as shown in b in FIG. 3.
   After a single EPM (7) molecule was inserted into the phospholipid bilayer (6) membrane, the voltage values on both sides of the EPM (7) nanopore were varied, the current values were read to plot the current-voltage (I-V) curve, and the conductance values of the individual EPM (7) nanopore were obtained by calculating the slope of the curve, and the results are shown in d in FIG. 3. The statistical conductance value of a single EPM (7) molecule when forming a nanopore was determined by reading the slope of the I-V curve multiple times or by plotting the statistical distribution with successive step jump interval values, as shown in c in FIG. 3.
   The current trajectories and normalized current histograms of individual EPM (7) nanopores and gating behavior in different concentrations of KCl solutions were recorded and compared in e in FIG. 3. a, c-e in FIG. 3 all demonstrate the successful formation of stable single-channel nanopore structures by EPM (7) molecules.

The application of synthetic macrocyclic compounds forming stable single-molecule nanopore structures on phospholipid bilayers (6) for potassium ion selectivity and peptide sequencing is illustrated by the following examples.
12) Potassium ion selectivity experiments with EPM (7) nanopores, wherein:
   Experiments on the selectivity of EPM (7) nanopores for potassium ions in solutions with different concentrations of KCl and NaCl:
   The experimental steps 7), 9) and 10) were repeated, where the solution injected in step 7) was 50 mM potassium chloride solution. After a single EPM (7) molecule was inserted into the phospholipid bilayer (6) membrane, the voltage values on both sides of the EPM (7) nanopore were changed, the current values were read to plot the I-V curve, and the slope of the I-V curve was calculated to obtain the conductance value of a single EPM (7) nanopore in 50 mM potassium chloride solution.
   The experimental steps 7), 9) and 10) were repeated, where the solution injected in step 7) was 50 mM sodium chloride solution. After a single EPM (7) molecule was inserted into the phospholipid bilayer (6) membrane, the voltage values on both sides of the EPM (7) nanopore were changed, the current values were read to plot the I-V curve, and the slope of the I-V curve was calculated to obtain the conductance value of a single EPM (7) nanopore in 50 mM sodium chloride solution.
   The conductance values of individual EPM (7) nanopores in 50 mM KCl solution were compared with those of individual EPM (7) nanopores in 50 mM NaCl solution to obtain the selectivity factor of EPM (7) nanopores for potassium ions in 50 mM solution.
   The above steps were repeated in 100 mM, 200 mM, 300 mM, 400 mM, 500 mM, 600 mM, 800 mM, 1000 mM, and 2000 mM solutions of potassium chloride or sodium chloride, and finally the conductivity values of individual EPM (7) nanopores obtained in the same concentration of potassium chloride solution and sodium chloride solution were compared to obtain the selectivity factors of EPM (7) nanopores for potassium ions in different concentrations of KCl and NaCl solutions. The results are shown in a-d in FIG. 4. The selectivity of EPM (7) nanopores for potassium ions in 2 M solution was up to 20-fold without additional modification.
   The potassium ion selectivity of EPM (7) is further illustrated by the following experiments on the potassium ion selectivity of EPM (7) nanopores in mixed solutions:
   Prepare a mixed solution with a total ionic strength of 500 mM, where the concentrations of potassium chloride and sodium chloride are: 500 mM potassium chloride plus 0 mM sodium chloride, 400 mM potassium chloride plus 100 mM sodium chloride, 250 mM potassium chloride plus 250 mM sodium chloride, 100 mM potassium chloride plus 400 mM sodium chloride, 0 mM potassium chloride plus 500 mM sodium chloride, respectively. Corresponding buffer solutions such as HEPES buffer solution, Tris-EDTA buffer solution can also be used as needed.
   The experimental steps 7), 9) and 10) were repeated, where the solution injected in step 7) was 500 mM potassium chloride plus 0 mM sodium chloride solution. After a single EPM (7) molecule was inserted into the phospholipid bilayer (6) membrane, the voltage values on both sides of the EPM (7) nanopore were changed, the current values were read to plot the I-V curve, and the slope of the I-V curve was calculated to obtain the conductance value of a single EPM (7) nanopore in 500 mM potassium chloride plus 0 mM sodium chloride solution.
   The above steps were repeated in 400 mM KCl plus 100 mM NaCl, 250 mM KCl plus 250 mM NaCl, 100 mM KCl plus 400 mM NaCl, and 0 mM KCl plus 500 mM NaCl mixed solutions, and the I-V curves of individual EPM (7) nanopores obtained from the five mixed solutions were compared to examine the selectivity of EPM (7) nanopores for potassium ions. The results are shown in e-f in FIG. 4. The conductance of EPM (7) nanopores increased with the increase of potassium ion concentration in the mixed solutions, further demonstrating the potassium ion selectivity of EPM (7).
   Experiments on the selectivity of EPM nanopores for potassium ions in different concentration gradient experiments:
   The experimental steps 7), 9), and 10) were repeated, where the solution injected in step 7) was 100 mM KCl solution on the cis side (4) and 500 mM KCl solution on the trans side (3). After a single EPM molecule was inserted into the phospholipid bilayer (6) membrane, the voltage values on both sides of the EPM (7) nanopore were changed and the current values were read to plot the I-V curve, and the voltage corresponding to when the current value was 0 was read value. The silver/silver chloride electrode (1) used for the measurement and the standard silver/silver chloride electrode (1) are measured in 100 mM KCl solution and 500 mM KCl solution, respectively, and finally the voltage value read in the I-V curve when the current value is 0 is subtracted from the electrode's self-potential difference to obtain the redox potential value of the EPM (7) nanopore in the KCl solution at that concentration gradient.
   The experimental steps 7), 9), and 10) were repeated, where the solution injected in step 7) was 100 mM NaCl solution on the cis side (4) and 500 mM NaCl solution on the trans side (3). After a single EPM molecule was inserted into the phospholipid bilayer (6) membrane, the voltage values on both sides of the EPM (7) nanopore were changed and the current values were read to plot the I-V curve, and the voltage corresponding to when the current value was 0 was read value. The silver/silver chloride electrode (1) used for the measurement and the standard silver/silver chloride electrode (1) are measured in 100 mM NaCl solution and 500 mM NaCl solution, respectively, and finally the voltage value read in the I-V curve when the current value is 0 is subtracted from the electrode's self-potential difference to obtain the redox potential value of the EPM (7) nanopore in the NaCl solution at that concentration gradient.
   The GHK equation was used to calculate the selectivity factor of EPM (7) nanopores for potassium ions by substituting the redox potential values of individual EPM (7) nanopores obtained in potassium chloride solution and sodium chloride solution in the same concentration gradient.
   Replacing the concentration gradients of 1 M KCl-100 mM KCl, 1 M NaCl-100 mM NaCl, 2 M KCl-200 mM KCl, 2 M NaCl-200 mM NaCl, and repeating the above steps to obtain different concentration gradients The selectivity factors of EPM (7) nanopores for potassium ions in different concentration gradients can be compared with the selectivity factors of EPM (7) nanopores for potassium ions obtained from experiments with different concentrations of KCl and NaCl solutions to verify the accuracy of the experimental results. The results are shown in a-c in FIG. 5, which further demonstrate the potassium ion selectivity of EPM.
   Experiments on the potassium ions selectivity of EPM nanopores in asymmetric solutions:
   The experimental steps 7), 9) and 10) were repeated, where the solution injected in step 7) was 1 M potassium chloride solution on the cis side (4) and 1 M sodium chloride solution on the trans side (3), and when a single EPM (7) molecule was inserted into the phospholipid bilayer (6) membrane, the voltage values on both sides of the EPM (7) nanopore were changed and the current values were read to plot the I-V curves, and the difference in potential values at positive and negative values was observed to examine the pores to check the selectivity of potassium ions. The results are shown in d in FIG. 5.
13) EPM nanopore applications for biomolecule detection or sequencing, proof-of-principle with peptide experiments, wherein:
   After repeating 7), 9) and (10) experimental steps to obtain stable single EPM (7) nanopores, the GG peptide short chain solution was then added to cis side (4), voltage was applied, current blocking events were observed and current traces were recorded, and the results are shown in a-b in FIG. 6. The voltage was changed again, current blocking events were observed and current traces were recorded, and the results are shown in c-d in FIG. 6.
   As each amino acid passes through the nanopore, the degree of ionic current alteration due to. a certain spatial blockage caused by the amino acid within the pore varies, as does the difference in the current blockage signal caused by the amino acid species. Given that there are as many as 20 amino acid species that make up a protein, five times more than the four bases used in DNA sequencing, for example, the MspA pore (Laszlo, A., Derrington, I., Ross, B. et al. Decoding long nanopore sequencing reads of natural DNA. Nat Biotechnol 32, 829-833 (2014). https://doi.org/10.1038/nbt.2950), four or more amino acids can be present in the pore at the same time, causing up to 20⁴ types of current blocking signals when proteins pass through the nanopore, so single-molecule protein sequencing using this conventional nanopore faces many difficulties. However, compared with the pore thickness of other transmembrane nanopore structures that have been attempted for protein sequencing, the pore thickness of the EPM nanopore disclosed in the present invention is only atomic level (6 Å, see FIG. 1), so the number of amino acids present in the pore at the same time can be theoretically reduced to one, which greatly improves the spatial resolution and provides great possibilities for single-molecule protein sequencing. method, the structure is uniform and can form a stable nanopore structure with good experimental reproducibility, so theoretically the method can be used for protein sequencing without technical barriers.
   After preliminary experiments, it was found that the current blocking phenomenon caused by protein peptide perforation could be observed in the EPM nanopore, which means it has the possibility to realize protein sequencing.
   Based on current developments, it has become possible to design larger cavity macrocyclic compounds and apply them to transmembrane nanopores. Nanopores formed by macrocyclic molecules with a pore size larger than 12 Åcan allow DNA single-stranded molecules to pass through for DNA single-stranded sequencing, and nanopores formed by macrocyclic molecules with a pore size larger than 24 Åcan be used for DNA double-stranded sequencing.
   The above mentioned is not a limitation of the present invention, and it should be noted that for a person of ordinary skill in the art, several variations, adaptations, additions or substitutions can be made without departing from the substantial scope of the present invention, for example, a rich selection of chemical mechanisms can provide other macrocyclic molecules with similar pore structures, pore size modulation by adding chemical groups, different side chain derivatization of macrocyclic molecule nanopores using rich modification chemistry, and other biological or chemical analyses based on the same principles using such nanopore structures, and these improvements and embellishments should also be considered within the scope of protection of the present invention.

1. An artificial synthetic macrocycle molecular nanopore structure, wherein the nanopore structure is a single-molecule transmembrane nanopore structure with nanometer-sized channels formed by insertion of a synthetic macrocyclic compound into a phospholipid bilayer membrane in an electrolyte solution; and the synthetic macrocyclic compound has a cavity pore size of 1 Å-50 Å in diameter, wherein the cavity of the synthetic macrocyclic compound has an atomic level thickness of 1 Å-30 Å.

2. The structure of claim 1, wherein the synthetic macrocyclic compound has side chains that facilitate insertion of the synthetic macrocyclic compound into the phospholipid bilayer membrane to forming a stable transmembrane structure.

3. The structure of claim 2, wherein the side chains are linked to a macrocycle of the synthetic macrocyclic compound by amide or ether bonds or carbon-carbon bonds.

4. The structure of claim 1, wherein the synthetic macrocyclic compound is one selected from the group consisting of a cucurbiturate derivative, a cyclodextrin derivative, a crown ether derivative, and a macrocyclic compound derivative consisting of an aromatic hydrocarbon.

5. The structure of claim 4, wherein the synthetic macrocyclic compound is a pillar[6]arene derivative, and the pillar[6]arene derivative has a molecular formula of C₃₇₄H₃₈₈N₄₀O₅₆ and a structural formula as follows:

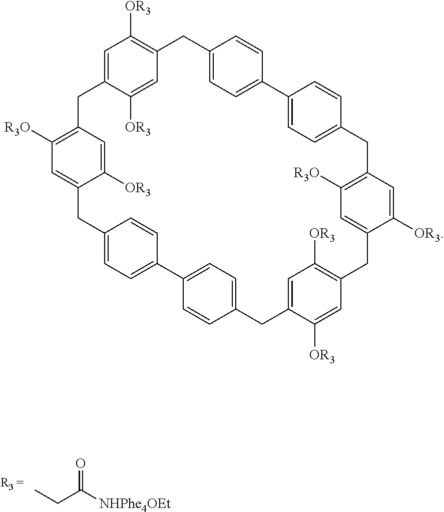

6. The structure of claim 5, wherein the synthetic steps of the structure comprise:
1) synthesizing the pillar[6]arene derivative by a chemical method;
2) preparing a perfusion cup for constructing the phospholipid bilayer membrane and performing ion channel experiments, wherein:
the perfusion cup is separated into a cis-side chamber and a trans-side chamber by a cup wall, wherein the cup wall has a support hole, and the phospholipid bilayer membrane is built on the support pore and then inserted into the synthetic macrocyclic compound to form the nanopore structure;
3) dissolving the synthetic macrocyclic compound in water or a buffer solution, sonicating, filtering undissolved material, dividing the filtered solution into aliquots, freezing, and storing the frozen aliquots;
4) polishing two pieces of silver wire with a sandpaper to remove an oxide layer on the surface of the silver wire, immersing the silver wire and a platinum electrode in a plating solution, the silver wire and the platinum electrode serving as the anode and cathode respectively, applying a voltage to prepare a silver/silver chloride electrode, and then connecting two silver/silver chloride electrodes to probes of a patch clamp instrument as anode and ground wire respectively;
5) preparing a lipid solution;
6) applying the lipid solution uniformly to both sides of the support hole of the perfusion cup using a brush until the support hole is uniformly covered and waiting for the lipids to dry at room temperature;
7) pipetting the electrolyte solution into each of the cis-side chamber and the trans-side chamber at a time;
8) performing the following steps in a Faraday box on an optical platform: immersing the silver/silver chloride electrodes serving as anode and ground wire in the electrolyte solution of the cis-side chamber and the trans-side chamber, respectively; and
turning on the patch clamp instrument, applying a positive potential to the trans side through the silver/silver chloride electrode, and grounding the cis side;
9) using a pipettor to lift a solution interface up and down on both sides of the support hole, such that a lipid monolayer formed by the lipid solutions on both sides forms a phospholipid bilayer membrane due to the hydrophobicity of hydrocarbon chains of the phospholipid molecules;
10) determining the phospholipid bilayer membrane as a bilayer structure by measuring the capacitance of the phospholipid bilayer membrane or by applying a membrane breaking voltage; and
11) thawing the frozen aliquots in step 3) by ultrasonication and then diluting the thawed aliquots using deionized water with 1 wt % of non-ionic surfactant; and
adding a solution of the synthetic macrocyclic compound very close to the support hole in the cis-side chamber, applying a voltage, and when a step jump in current occurs, it indicates that the synthetic macrocyclic compound has formed stable nanopore channels in the phospholipid bilayer membrane.

7. The structure of claim 6, wherein an outer chamber of the perfusion cup is provided with small holes connected to its inside chamber;
the lipid solution is one selected from the group consisting of 1,2-diacetyl-sn-glycero-3-phosphocholine, palmitoyl oleoyl phosphatidylcholine, dipalmitoyl phosphatidylcholine, and distearoyl phosphatidylglycerol dissolved in decane;
in step 10), a capacitance value is 35-90 pF when the phospholipid bilayer membrane is a bilayer structure; or when the phospholipid bilayer membrane is a bilayer structure, the phospholipid bilayer membrane is broken within an applied potential of 300-400 mV; and
in step 11), avoiding any air bubbles when adding a solution of synthetic macrocyclic compound; and when a step jump in current occurs, the voltage is reduced in time.

8. The structure of claim 6, wherein the perfusion cup and the cup wall are made of polyformaldehyde resin, polytetrafluoroethylene, or polystyrene.

9. The structure of claim 1, wherein the synthetic macrocycle molecular nanopore structure is used for efficient selective transport and separation of potassium ion/sodium ion.

10. The structure of claim 1, wherein the synthetic macrocycle molecular nanopore structure is used for protein peptide sequencing, or detection and sequencing of similar biomolecule and chemical molecule based on the same principle.

11. The structure of claim 1, wherein the synthetic macrocycle molecular nanopore structure is used for DNA sequencing or RNA sequencing.
